(19) United States
(12) Patent Application Publication
(10) Pub. No.: US 2008/0280338 A1
(43) Pub. Date: Nov. 13, 2008

(54) Biofuel Processing System

(76) Inventors: Kenneth R. Hall, College Station, TX (US); Mark T. Holtzapple, College Station, TX (US); Sergio C. Capareda, College Station, TX (US)

Correspondence Address: BAKER BOTTS L.L.P., 2001 ROSS AVENUE, SUITE 600, DALLAS, TX, 75201-2980, US

(21) Appl. No.: 12/118,484

(22) Filed: May 9, 2008

Related U.S. Application Data
(60) Provisional application No. 60/917,467, filed on May 11, 2007.

(51) Int. Cl. C12P 7/06; C12M 1/00

(52) U.S. Cl. 435/161; 435/289.1

(57) ABSTRACT

According to one embodiment, a biofuel processing system includes a biomass conversion system, a gasification reactor and/or a pyrolysis reactor, and a synthetic fuel creation system. The biomass conversion system uses a biological process to create a low-molecular-weight hydrocarbon stream from a biomass. The reactor generates heat and hydrogen using fresh biomass or undigested biomass from the biomass conversion system in which a portion of the heat is used by the biomass conversion system. The synthetic fuel creation system converts the low-molecular-weight hydrocarbon stream from the biomass conversion system and/or the reactor to liquefied fuel using another portion of heat from the reactor.

### RELATED APPLICATIONS

This application claims priority to U.S. Provisional Patent Application Ser. No. 60/917,467, entitled “BIOFUEL PROCESSING SYSTEM,” which was filed on May 11, 2007.

### TECHNICAL FIELD OF THE DISCLOSURE

This disclosure generally relates to biofuels, and more particularly, to a biofuel processing system for the production of biofuels from a biomass.

### BACKGROUND OF THE DISCLOSURE

Biological matter that has been converted to liquefied fuel is generally referred to as biofuel. Biofuel processes that create these biofuels typically use biological processing methods that produce alcohols, such as ethanol. Although these alcohols may have relatively high octane ratings, they have several disadvantages. For example, alcohols have a relatively lower energy density than other hydrocarbons, such as gasoline. Their relatively strong polarity increases the vapor pressure of fuels when added as a constituent such that air pollution is increased. Alcohols also have a tendency to absorb water. This may be problematic when shipping low-molecular-weight alcohols, such as ethanol, in common-carrier pipelines that may contain water. Ethanol is also corrosive and thus may damage pipelines or dissolve fiberglass fuel tanks.

### SUMMARY OF THE DISCLOSURE

According to one embodiment, a biofuel processing system includes a biomass conversion system, a gasification reactor and/or a pyrolysis reactor, and a synthetic fuel creation system. The biomass conversion system uses a biological process to create a low-molecular-weight hydrocarbon stream from a biomass. The gasification reactor generates heat and hydrogen using fresh biomass or undigested biomass from the biomass conversion system in which a portion of the heat is used by the biomass conversion system. The synthetic fuel creation system converts the low-molecular-weight hydrocarbon stream from the biomass conversion system and/or the pyrolysis reactor to liquefied fuel using another portion of heat from the gasification reactor.

Some embodiments of the disclosure provide numerous technical advantages. Some embodiments may benefit from some, none, or all of these advantages. For example, according to one embodiment, a fuel may be produced having a relatively high energy density that may be generally compatible with commonly used fuels, such as gasoline or kerosene. The biomass processing system includes a number of processing steps that may enable conversion of a relatively large portion of the energy content of the biomass ingredient. The efficiency of the conversion process may be enhanced by utilizing heat and/or mass from one process as an ingredient to another process. Thus, the biomass processing system may enable a relatively high degree of yield in relation to the amount of biomass introduced into the biofuel processing system.

Other technical advantages may be readily ascertained by one of ordinary skill in the art.

### BRIEF DESCRIPTION OF THE DRAWINGS

A more complete understanding of embodiments of the disclosure will be apparent from the detailed description taken in conjunction with the accompanying drawings in which:

FIG. 1 is a diagram showing one embodiment of a biofuel processing system according to the teachings of the present disclosure;

FIG. 2A is one embodiment of the biomass conversion system of FIG. 1 that converts biomass to secondary alcohols;

FIG. 2B is another embodiment of the biomass conversion system of FIG. 1 that converts biomass to primary alcohols;

FIG. 2C is another embodiment of the biomass conversion system of FIG. 1 that converts biomass to secondary alcohols;

FIG. 2D is another embodiment of the biomass conversion system of FIG. 1 that converts biomass to primary alcohols; and

FIG. 2E is another embodiment of the biomass conversion system of FIG. 1 that converts biomass to methane.

### DETAILED DESCRIPTION OF EXAMPLE EMBODIMENTS

As described above, conversion of biological matter into various types of alcohols has several disadvantages. To remediate these problems, various biological processing approaches have been developed in which the biomass is gasified to a synthesis gas from which other alcohols or hydrocarbons are created. One such process is a Fischer-Tropsch process that generates high-molecular-weight hydrocarbons from biomass. Known implementations of the Fischer-Tropsch process, however, generate syngas as an intermediary step, the processing of which may be capital intensive and generally energy inefficient.

FIG. 1 shows one embodiment of a biofuel processing system 10 that may provide a solution to this problem and other problems. Biofuel processing system 10 includes a biomass conversion system 12, a gasification and/or pyrolysis reactor referred to herein as reactor 14, and a synthetic fuel creation system 16 coupled as shown. Biomass conversion system 12 receives a biomass feed 18 and converts the biomass feed 18 to a low-molecular-weight hydrocarbon stream 20. Reactor 14 receives an undigested biomass stream 26 from biomass conversion system 12 and converts undigested biomass stream 26 to another low-molecular-weight hydrocarbon stream 22. These low-molecular-weight hydrocarbon streams 20 and 22 are fed to synthetic fuel creation system 16, which converts these low-molecular-weight hydrocarbon streams 20 and 22 to a liquified fuel 24, such as gasoline or other generally high-molecular-weight fuel. Certain embodiments of biofuel processing system 10 may generate liquified fuel 24 that may not have the previously cited drawbacks of other relatively low-molecular-weight alcohols, such as ethanol.

Biomass conversion system 12 receives any suitable form of organic matter and generates various low-molecular-weight hydrocarbons, such as alcohol or methane using a biological process. Suitable forms of organic matter may include municipal solid waste (MSW), sewage sludge, manure, or plantstuffs, such as algae, crop residues, or energy crops. In one embodiment, biomass conversion system 12 may include biological cultures that promote the decomposition of biomass feed 18 using a fermentation process for the production of alcohols, such as ethanol. In another embodiment, biomass conversion system 12 may include biological cultures that promote the decomposition of biomass feed 18 using a digester process for the production of methane. In another embodiment, biomass conversion system 12 may include a fermentation process and a digester process that coexist with one another. That is, a fermentation process and a digester process may be integrated within biomass conversion system 12 to generate alcohol and methane, respectively, that synthetic fuel creation system 16 uses to generate liquefied fuel 24.

Certain embodiments incorporating an integral fermentation and digester process may reduce filtering of the biomass feed 18 prior to processing by biomass conversion system 12. Particular types of biomass, such as grain sorghum or corn, may include glucose that is generally more conducive to decomposition using the fermentation process. Conversely, other types of biomass, such as those containing cellulose may be relatively more conducive to decomposition using a digester process. Selective separation or filtering of these types of biomass may not be required by biomass conversion system 12 due to its integral fermentation and digester process. In some embodiments therefore, biomass conversion system 12 may operate at a reduced cost relative to known biofuel processing systems, such as those described above.

Reactor 14 generates heat 26, a hydrogen stream 30, a water stream 36, char 38, and waste gases 40 from undigested biomass stream 26 by reacting undigested biomass 26 at a relatively high temperature with a controlled amount of oxygen. In one embodiment, the hydrogen stream 30 may be used to generate additional heat 28 for biomass conversion system 12 and/or synthetic fuel creation system 16. In another embodiment, a hydrogen stream 30 may be transmitted to biomass conversion system 12 to produce alcohols from intermediate chemicals. In some embodiments, heat 28 may also include waste heat from the gasification process. Waste heat generally refers to excess thermal energy generated by reactor 14. This waste heat may be used for other processes, such as biomass conversion system 12 and/or synthetic fuel creation system 16.

In an embodiment in which reactor 14 includes a pyrolyzer reactor, the pyrolyzer that pyrolyzes the undigested biomass stream 26 to form the water stream 36 and low-molecular-weight hydrocarbon stream 22. The pyrolyzer may reduce the relative amount of char 38, waste gas 40, or waste gas 32 produced by biofuel processing system 10. Waste from the reactor 14 may be emitted as char 38 and waste gas 40. The pyrolyzer is generally operable to convert most forms of biomass into streams that can be converted into useable energy. Pyrolyzer may accept various forms of biomass similarly to biomass conversion system 12 as well as other non-biodegradable components of biomass feed, such as plastics. Water stream 36 may be transferred to biomass conversion system 12 and/or synthetic fuel creation system 16. In some regions in which access to water may be scarce, water stream 36 may be diverted to other systems. An additional hydrocarbon stream 20 may be transferred to synthetic fuel creation system 16 for production of liquified fuel 24. Principally, the pyrolyzer can convert the lignin content of the biomass into hydrocarbons that the synthetic fuel creation process 16 can convert into conventional fuels.

In biomass conversion system 12, the easy-to-digest portions of biomass feed 18 are processed first, leaving the hard-to-digest portions for reactor 14. Processing the biomass feed 18 to a high conversion rate by biomass conversion system 12 may require a relatively long residence time. For example, to achieve approximately 80 percent conversion of the biomass feed 18 in biomass conversion system 12 typically requires approximately 3 months, whereas 70 percent conversion may require approximately 2 months. Thus in one embodiment, biomass conversion system 12 may have may a conversion rate of biomass feed 18 to low-molecular-weight hydrocarbon stream 20 that is less than 70 percent. Incorporation of the reactor 14 having a pyrolyzer for processing of undigested biomass stream 26 may provide a relatively shorter residence time in biomass conversion system 12. Reactor 14 may also reduce the amount of residue in the form of waste gas 32, char 38, waste gas 40 generated by biofuel processing system 10 in some embodiments.

The product spectrum of reactor 14 depends upon how it operates. If the oxygen:biomass ratio is high, the products favor carbon monoxide and hydrogen with less char 38. Unfortunately, because of the high oxygen usage, a greater portion of the biomass energy is lost as heat and relatively more cost may be associated with producing the oxygen. If the oxygen:biomass ratio is low, relatively more hydrocarbons and char may be formed. Thus, the oxygen:biomass ratio may be tailored to suit various types of operating conditions of biofuel processing system 10.

Synthetic fuel creation system 16 creates liquid fuel 24, such as gasoline, jet fuel, and/or diesel and a waste gas stream 46 from low-molecular-weight hydrocarbon streams 20 and 22. In one embodiment, synthetic fuel creation system 16 includes a relatively high temperature cracker that converts low-molecular-weight hydrocarbons, such as methane, into acetylene and hydrogen. After quenching, the acetylene and a portion of the hydrogen are converted catalytically into ethylene. The ethylene passes over an oligomerization catalyst to produce liquid fuel 24, which may be, for example, gasoline, jet fuel, diesel, or a fuel mix. The same catalyst may also convert alcohols from hydrocarbon streams 20 and 22 to liquid fuel 24. Synthetic fuel creation system 16 may generate a hydrogen stream 44 that may be fed to biomass conversion system 12. In one embodiment, synthetic fuel creation system 16 may also generate a recycle gas stream 42 that may be used by reactor 14.

Certain embodiments incorporating synthetic fuel creation system 16 may provide an advantage in that in the event that biomass feed 18 is not available because of storms, drought, disease, or an upset in the fermentation, synthetic fuel creation system 16 can process natural gas into fuels or chemicals until the fermentation is again available.

The ability to oligomerize alcohols into alkanes, such as jet propellant 8 (JP-8), has been demonstrated by the Mobil methanol-to-gasoline process, which was commercialized in New Zealand. The following is the stoichiometry for methanol to nonane:

H₂+9H₃COH→C₉H₂O+9H₂O

The energy retained in the final product is calculated from the heats of combustion:

$$Energy Retained = \frac{( 1 mol nonane ) ( 6124.5 \frac{kJ}{mol} )}{\begin{matrix}( 1 mol hydrogen ) ( 285.84 \frac{kJ}{mol} ) + \\ ( 9 mol methanol ) ( 726.6 \frac{kJ}{mol} )\end{matrix}} = 89.7 \%$$

The mass retained in the final product is:

$$Mass Retained = \frac{( 1 mol nonane ) ( \frac{128.25 g}{mol} )}{\begin{matrix}( 1 mol hydrogen ) ( \frac{2.016 g}{mol} ) + \\ ( 9 mol methanol ) ( \frac{32.04 g}{mol} )\end{matrix}} = 44.2 \%$$

The following is the stoichiometry for ethanol to octane:

H₂+4H₃CCH₂OH→C₈H₁₈+4H₂O

The energy retained in the final product is calculated from the heats of combustion:

$$Energy Retained = \frac{( 1 mol octane ) ( 5470.7 \frac{kJ}{mol} )}{\begin{matrix}( 1 mol hydrogen ) ( 285.84 \frac{kJ}{mol} ) + \\ ( 4 mol ethanol ) ( 1366.9 \frac{kJ}{mol} )\end{matrix}} = 95.1 \%$$

The mass retained in the final product is:

$$Mass Retained = \frac{( 1 mol octane ) ( \frac{114.22 g}{mol} )}{\begin{matrix}( 1 mol hydrogen ) ( \frac{2.016 g}{mol} ) + \\ ( 4 mol ethanol ) ( \frac{46.07 g}{mol} )\end{matrix}} = 61.3 \%$$

The following is the stoichiometry for isopropanol to nonane:

H₂+3H₃CCHOHCH₃→C₉H₂O+3H₂O

The energy retained in the final product calculated from the heat of combustion is:

$$Energy Retained = \frac{( 1 mol nonane ) ( 6124.5 \frac{kJ}{mol} )}{\begin{matrix}( 1 mol hydrogen ) ( 285.84 \frac{kJ}{mol} ) + \\ ( 3 mol isopropanol ) ( 1986.6 \frac{kJ}{mol} )\end{matrix}} = 98.1 \%$$

The mass retained in the final product is:

$$Mass Retained = \frac{( 1 mol nonane ) ( \frac{128.25 g}{mol} )}{\begin{matrix}( 1 mol hydrogen ) ( \frac{2.016 g}{mol} ) + \\ ( 3 mol methanol ) ( \frac{60.09 g}{mol} )\end{matrix}} 70.4 \%$$

The calculations described above show that oligomerizing higher alcohols may retain a relatively larger percentage of the alcohol energy in the alkane product. This may not be the case with lower alcohols. Additionally, a greater fraction of the mass may be retained when oligomerizing higher alcohols.

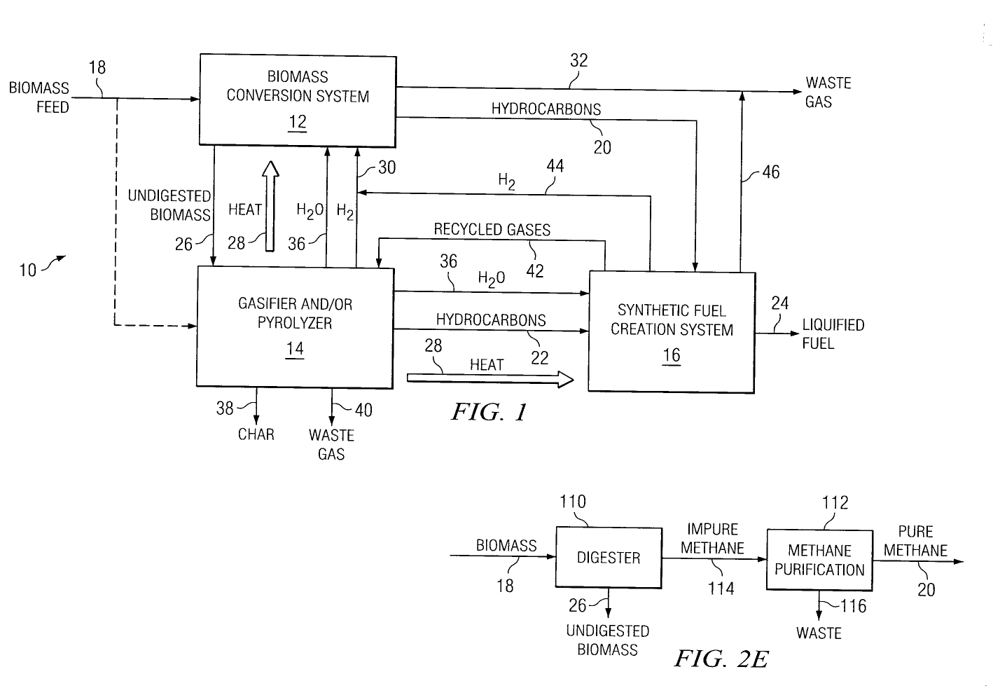

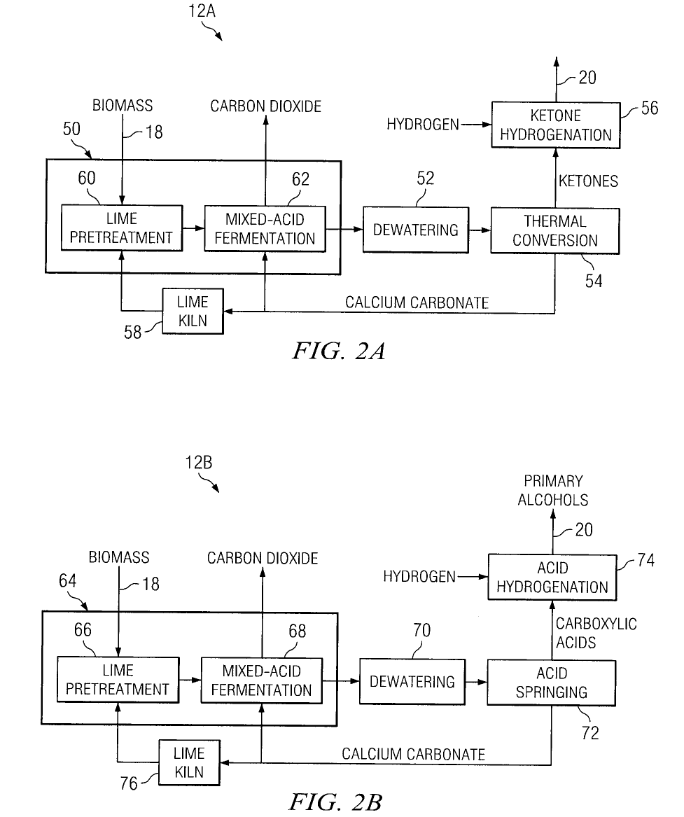

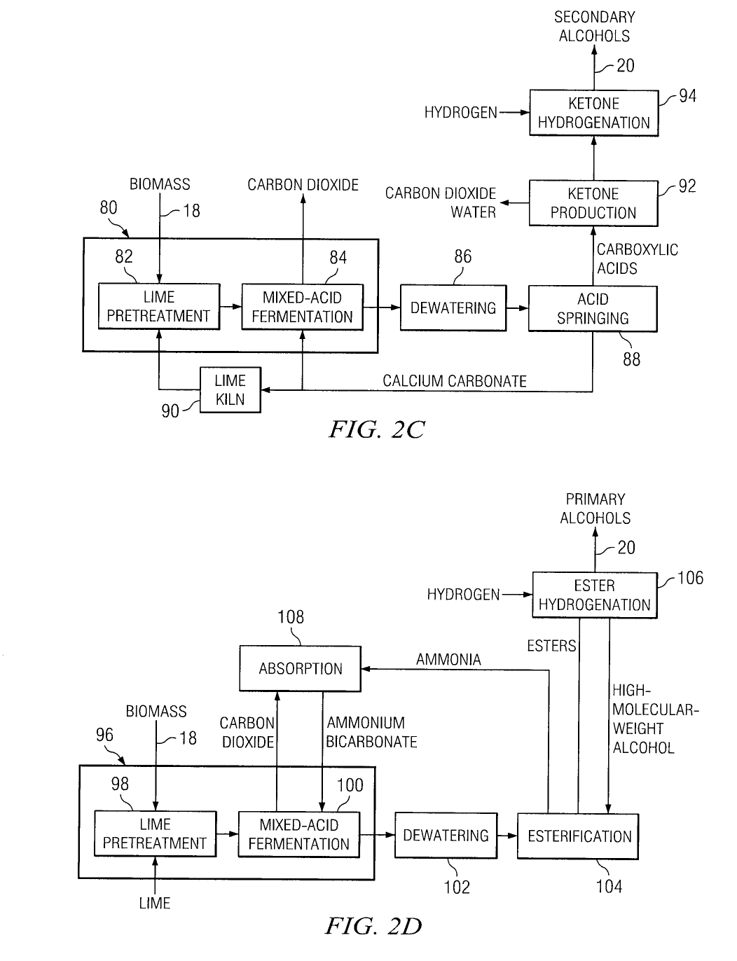

Biomass conversion system 12 may include any system for converting biomass into a mixture of alcohols. FIGS. 2A through 2D show various embodiments of biomass conversion systems 12 that may be used with the biomass processing system 10 of the present disclosure.

FIG. 2A shows one embodiment of a biomass conversion system 12A that may be used to generate low-molecular-weight hydrocarbon stream 20 including secondary alcohols. Biomass conversion system 12A generally includes a lime treatment section 50, a dewatering section 52, a thermal conversion section 54, a ketone hydrogenation section 56, and a lime kiln 58 coupled as shown. Lime treatment section 50 includes a lime pretreatment portion 60 and a mixed-acid fermentation portion 62. Lime pretreatment portion 60 mixes the incoming biomass feed 18 with lime from lime kiln 58 to enhance its digestibility. The lime-treated biomass is then fermented in mixed-acid fermentation section 62 using a mixed-culture of microorganisms that produces a mixture of carboxylic acids, such as acetic acid, propionic acid, and/or butyric acid. Calcium carbonate may be added to mixed-acid fermentation portion 62 to neutralize the acids to form their corresponding carboxylate salts, such as calcium acetate, calcium propionate, and calcium butyrate. After fermentation, these salts may be converted thermally to ketones in dewatering section 52 and thermal conversion section 54. Ketone hydrogenation section 56 may be used to catalytically hydrogenate the ketones into secondary alcohols, such as isopropanol.

FIG. 2B shows another embodiment of biomass conversion system 12B that may be used to generate low-molecular-weight hydrocarbon stream 20 comprising primary alcohols. Biomass conversion system 12B includes a lime treatment section 64 having a lime pretreatment portion 66 and a mixed-acid fermentation portion 68, a dewatering section 70, a acid springing section 72, an acid hydrogenation section 74, and a lime kiln 76 coupled as shown. Lime treatment section 64, dewatering section 70, and lime kiln 76 function in a manner similar to lime treatment section 50, dewatering section 52, and lime kiln 58 of biomass conversion system 12A. Biomass conversion system 12B differs, however, in that acid springing section 72 springs carboxylic acids from the concentrated carboxylate salt solution. In the acid springing step, carboxylate salts react with a tertiary amine and carbon dioxide causing calcium carbonate to precipitate while amine carboxylate remains in solution. In a reactive distillation column, the amine carboxylate thermally cracks into tertiary amine and carboxylic acid. The tertiary amine and calcium carbonate are recycled within the process consuming relatively few chemicals. The resulting acids react with a high-molecular-weight alcohol, such as heptanol, to form the corresponding esters. In the acid hydrogenation section 74, the esters are hydrogenated to form primary alcohols. The high-molecular-weight alcohol is recovered by distillation and the low-molecular-weight primary alcohols are transported to synthetic fuel creation system 16.

FIG. 2C shows another embodiment of biomass conversion system 12C that may convert the biomass to low-molecular-weight hydrocarbon stream 20 comprising secondary alcohols. Biomass conversion system 12B includes a lime treatment section 80 having a lime pretreatment portion 82 and a mixed-acid fermentation portion 84, a dewatering section 86, an acid springing section 88, and a lime kiln 90 similarly to biomass conversion system 12B of FIG. 2B. Biomass conversion system 12C differs, however, in that it includes a ketone production section 92 and a ketone hydrogenation section 94. Ketone production section 92 catalytically converts carboxylic acids into ketones, which are subsequently hydrogenated by ketone hydrogenation section 94 into secondary alcohols that may be included in the low-molecular-weight hydrocarbon stream 20.

FIG. 2D shows another embodiment of biomass conversion system 12D that may convert the biomass feed 18 to low-molecular-weight hydrocarbon stream 20 comprising primary alcohols. Biomass conversion system 12D includes a lime treatment section 96 having a lime pretreatment portion 98 and a mixed-acid fermentation portion 100, a dewatering section 102, an esterification section 104, an ester hydrogenation section 106, and an absorption section 108. Lime pretreatment portion 98 mixes the incoming biomass feed 18 with lime to enhance its digestibility. The pretreated biomass is then fed to mixed-acid fermentation section 100 where a mixed culture of microorganisms produces mixed acids that are neutralized with an ammonium bicarbonate stream from absorption section 108.

The ammonium salts are concentrated and then esterified in esterificaton section 104 by adding a high-molecular-weight alcohol, which releases ammonia. The ammonia is recovered in absorber section 108 where it reacts with carbon dioxide to produce ammonium bicarbonate. The esters are hydrogenated to produce primary alcohols. The high-molecular-weight alcohol is recycled in esterification section 104, and the low-molecular-weight alcohols are transmitted to synthetic fuel creation system 16.

The molecular weight distribution of the low-molecular-weight hydrocarbon stream 20 depends upon operating temperatures and the amount of buffer used. Lower temperatures, (e.g., 40 degrees Celsius) may favor higher alcohols while higher temperatures (e.g., 55 degrees Celsius) may favor lower alcohols. Calcium carbonate buffer may favor higher alcohols while ammonium bicarbonate buffer may favor lower alcohols.

FIG. 2D shows another embodiment of biomass conversion system 12E that may convert the biomass feed 18 to low-molecular-weight hydrocarbon stream 20 comprising relatively pure methane. Biomass conversion system 12E generally includes a digester 110 and a methane purification process 112 as shown. Digester 110 receives biomass stream 18 and produces an impure methane stream 114 and undigested biomass stream 26 that may be fed to gasifier 14. Methane purification process 112 filters waste from impure methane stream 114 to form low-molecular-weight hydrocarbon stream 20 including relatively pure methane that is fed to synthetic fuel creation system 16. The waste may be emitted from methane purification process 112 as waste stream 116. This biomass conversion process 12E may avoid the production of any significant amounts of alcohols by producing mainly methane, which may be used by synthetic fuel creation system 16 for the production of high-molecular-weight alcohols.

A biofuel processing system 10 has been described that may provide enhanced efficiency as well as other benefits over other known biofuel processing systems. This is accomplished using the synergies provided by the combination of biomass conversion system 12, reactor 14, and synthetic fuel creation system 16. For example, Waste heat from the reactor 14 can be used as an energy source to run the other portions of the plant. As another example, the mixed culture of microorganisms in the biomass conversion system 12 contains methanogens. To limit methane production, inhibitors are added to suppress the methanogens. If the inhibition is imperfect, the resulting methane can be sent to the synthetic fuel creation system 16 and converted to liquid fuel 24.

In alternative embodiments, biomass conversion system 12 may be operated without any inhibitors, which would produce primarily methane and no alcohols. The methane from biomass conversion system 12 after polishing to remove undesirable components could then be sent to the synthetic fuel creation system 16 to make liquid hydrocarbons. This process has the advantage of eliminating the downstream processing steps in the biomass conversion system 12 in some embodiments.

Although the present disclosure has been described with several embodiments, a myriad of changes, variations, alterations, transformations, and modifications may be suggested to one skilled in the art, and it is intended that the present disclosure encompass such changes, variations, alterations, transformation, and modifications as they fall within the scope of the appended claims.

What is claimed is:

1. A biofuel processing system comprising:
a biomass conversion system that is operable to create one of an alcohol stream and a methane stream from a biomass using a biological process;
a reactor operable to:
receive an undigested biomass from the biomass conversion system, the undigested biomass comprising a portion of the biomass not used to create the alcohol and methane stream; and
generate heat from the undigested biomass, a portion of the heat transmitted to the biomass conversion system;
create an additional one of an alcohol stream and a methane stream the undigested biomass; and
a synthetic fuel creation system operable to:
receive the one of an alcohol stream and a methane stream from the biomass conversion system and the additional one of an alcohol stream and a methane stream from the reactor;
receive another portion of the heat generated by the reactor; and
convert methane present in the alcohol and methane stream and the additional alcohol and methane stream into acetylene and hydrogen;
hydrogenate the acetylene and the hydrogen to ethylene; and
oligermerize the ethylene or alcohols present in the one of the alcohol and methane stream and the additional one of the alcohol and methane stream to liquefied fuel.

2. A biofuel processing system comprising:
a biomass conversion system that is operable to create one of an alcohol stream and a methane stream from a biomass using a biological process;
a reactor operable to:
receive an undigested biomass from the biomass conversion system, the undigested biomass comprising a portion of the biomass not used to create the low-molecular-weight hydrocarbon stream; and
generate heat from the undigested biomass, a portion of the heat transmitted to the biomass conversion system; and
a synthetic fuel creation system operable to:
receive the one of an alcohol stream and a methane stream from the biomass conversion system;
receive another portion of the heat generated by the reactor; and
convert the one of an alcohol stream and a methane stream to liquefied fuel.

3. The biofuel processing system of claim 2, wherein the reactor further comprises a pyrolyzer that is operable to create an additional one of an alcohol stream and a methane stream from the undigested biomass, the synthetic fuel creation system operable to convert the additional one of an alcohol stream and a methane stream to the liquefied fuel.

4. The biofuel processing system of claim 2, wherein the synthetic fuel creation system is operable to receive the one of an alcohol stream and a methane stream comprising alcohol or methane from the biomass conversion system.

5. The biofuel processing system of claim 2, wherein at least a portion of the heat generated by the reactor comprises waste heat that is used by the biomass conversion system.

6. The biofuel processing system of claim 2, wherein the reactor is operable to generate the heat by creating hydrogen (H2) and carbon-monoxide (CO) and burning the hydrogen and carbon-monoxide.

7. The biofuel processing system of claim 2, wherein the synthetic fuel creation system is operable to convert the one of an alcohol stream and a methane stream to liquefied fuel by:
converting methane present in the one of an alcohol stream and a methane stream to acetylene and hydrogen;
hydrogenating the acetylene and the hydrogen to ethylene; and
oligermerizing the ethylene and alcohols present in the one of an alcohol stream and a methane stream to the liquefied fuel.

8. The biofuel processing system of claim 2, wherein the synthetic fuel creation system comprises a Fischer-Tropsch process.

9. The biofuel processing system of claim 2, wherein less than 70 percent of the biomass feed is converted to low-molecular-weight hydrocarbons in the biomass conversion system.

10. The biofuel processing system of claim 2, wherein biomass conversion system is operable to receive hydrogen from the reactor.

11. The biofuel processing system of claim 2, wherein the biomass conversion system is operable to create the one of an alcohol stream and a methane stream comprising secondary alcohols using a hydrogenation process.

12. The biofuel processing system of claim 2, wherein the biomass conversion system is operable to create the one of an alcohol stream and a methane stream comprising primary alcohols by esterifying the biomass and hydrogenating the esterified biomass to the primary alcohols.

13. A method comprising:
creating one of an alcohol stream and a methane stream from a biomass using a biological process;
generating heat using a gasification process that processes undigested biomass from the biological process, a portion of the heat used to create the one of an alcohol stream and a methane stream; and
converting the one of an alcohol stream and a methane stream to liquefied fuel using another portion of the heat.

14. The method of claim 13, further comprising creating another one of an alcohol stream and a methane stream from the undigested biomass using a pyrolyzer, the gasification process comprising the pyrolyzer.

15. The method of claim 13, wherein creating the one of an alcohol stream and a methane stream further comprises creating alcohol or methane from the biomass.

16. The method of claim 13, wherein generating the heat further comprises generating waste heat as a byproduct of the gasification process.

17. The method of claim 13, generating the heat further comprises heat by creating hydrogen (H2) and carbon-monoxide (CO) and burning the hydrogen and carbon-monoxide.

18. The method of claim 13, wherein converting the one of an alcohol stream and a methane stream to liquefied fuel further comprises converting methane present in the one of an alcohol stream and a methane stream to acetylene, hydrogenating the acetylene to ethylene, and oligermerizing the ethylene and alcohols present in the one of an alcohol stream and a methane stream to the liquefied fuel.

19. The method of claim 13, wherein converting the one of an alcohol stream and a methane stream to liquefied fuel further comprises converting the one of an alcohol stream and a methane stream to liquefied fuel using a Fischer-Tropsch process.

20. The method of claim 13, wherein creating the one of an alcohol stream and a methane stream from the biomass using the biological process further comprises creating the one of an alcohol stream and a methane stream from less than 70 percent of the biomass using the biological process.

21. The method of claim 13, wherein generating heat using the gasification process further comprises generating hydrogen using the gasification process that is used by the biological process.

22. The method of claim 13, wherein creating the one of an alcohol stream and a methane stream, further comprises creating the one of an alcohol stream and a methane stream comprising secondary alcohols, the biological process comprising a hydrogenation process.

23. The method of claim 13, wherein creating the one of an alcohol stream and a methane stream, further comprises creating the one of an alcohol stream and a methane stream comprising primary alcohols by esterifying the biomass and hydrogenating the esterified biomass to the primary alcohols.
